(19) United States
(12) Patent Application Publication
(10) Pub. No.: US 2010/0184219 A1
(43) Pub. Date: Jul. 22, 2010

(54) Methods and Apparatuses Relating to Cell Culture Media

(75) Inventor: Nicholas Robert Forsyth, Stoke on Trent (GB)

Correspondence Address: SWANSON & BRATSCHUN, L.L.C., 8210 SOUTHPARK TERRACE, LITTLETON, CO, 80120, US

(73) Assignee: KEELE UNIVERSITY, Keele (GB)

(21) Appl. No.: 12/666,663

(22) PCT Filed: Jul. 7, 2008
(86) PCT No.: PCT/GB08/02304

§ 371 Date: Feb. 1, 2010

(30) Foreign Application Priority Data
Jul. 6, 2007 (GB) 0713121.2

(51) Int. Cl. C12N 5/02; C12M 1/38; C12M 1/36; C12M 1/00

(52) U.S. Cl. 435/383; 435/404; 435/286.1; 435/286.6; 435/289.1

(57) ABSTRACT

A method of preparing cell culture media for use in culturing cells and an apparatus for carrying out the method. The method includes providing a vessel containing cell culture media and exposing the cell culture media to a first controlled atmosphere having a predetermined O₂ concentration that is less than an external atmospheric O₂ concentration, to reduce the O₂ concentration in the cell culture media before culturing cells in the cell culture media. The cell culture media may be agitated during exposure to the first controlled atmosphere. The temperature and/or pressure of the first controlled atmosphere may be controlled. Also provided is cell culture media which has been prepared by the method, and also a method of culturing cells in cell culture media prepared by the method

The invention relates to the field of in vitro cell culture, and the biological growth media used for cell culture, commonly referred to as cell culture media. In particular, the invention relates to the preparation of cell culture media before culturing cells in the cell culture media.

In vitro culture of cells and tissues requires a controlled environment with both temperature and gas concentrations to which cell culture media is exposed being carefully regulated. Because most cell culture media use a bicarbonate-based buffering system, incubators used for cell culture routinely provide a controlled gas environment enriched in carbon dioxide (CO₂), which is commonly maintained at 5% (as opposed to a normal external atmospheric concentration of about 0.036%). Culture flasks usually allow equilibration of gas concentrations between the flask contents and the incubator by means of gas-permeable stoppers of various types, although, for some purposes, individual flasks may be flushed with 5% CO₂ and sealed. By this means, the cell culture media not only provides the necessary nutrients, salts, sugars, growth factors and substratum, but also maintains the pH at the appropriate level for optimal cell growth. The required pH is 7.4, although the exact value may vary for specific cell types or experimental requirements. Different buffer concentrations or types may also require different concentrations of CO₂ in order to maintain the required pH.

CO₂ levels are increased in cell culture incubators by the introduction of excess CO₂ which displaces air allowing the adjustment of gas concentration. The addition of CO₂ is controlled by means of a CO₂ sensor linked to an automatic system that adds more CO₂ or vents the incubator to purge CO₂ as required.

Recent discoveries have demonstrated that a further component of the gas phase composition is also important for in vitro culture of cells. Cells cultured in, for example, 2% O₂ (as opposed to the normal atmospheric concentration of 21%) display reduced DNA damage, altered size/shape, enhanced chromosomal stability, and an increased proliferative potential [1,2]. This reduced O₂ concentration better reflects the partial O₂ concentration (pO₂) of tissues under normal physiological conditions, since most such tissues are not exposed to O₂ at normal external atmospheric concentrations of O₂. Indeed, at a cellular level, O₂ can be a significant toxin. Tissues are exposed to a wide range of O₂ concentrations depending on their anatomical location, even within a single organ, depending on perfusion. For instance, oxygen tensions within the liver vary from about 4% in periportal regions of the liver lobules to about 2% for tubular cells of the renal papilla [3].

Hypoxia (a deficiency of O₂) is known to be an inducer of large numbers of genes, not restricted only to those directly involved in the physiological response to hypoxia, such as those required for haematopoiesis and angiogenesis [4,5]. It is increasingly clear that for the in vitro culture of many cell types, consistent exposure to O₂ concentrations well below normal external concentrations is important. For instance fetal lung fibroblasts, embryonic stem cells, chondrocytes, mesenchymal stem cells, and haematopoetic stem cells [2,6-10].

Cell culture incubators which provide control of both CO₂ and O₂ levels are known. However, it takes approximately 30 minutes for an Oxygen-controlled incubator to displace sufficient air to create a 5% O₂ environment. Within a 50 L incubator it takes a minimum of 180 minutes to create a 5% O₂ headspace in 25 cm² and 420 minutes in a 150 cm² perforated lid flasks. This situation is slightly less severe in a modular ‘sealed flask’ system where approximately 10 minutes are required to drive all oxygen out of a 75 cm² flask [11].

These deficiencies in Oxygen-controlled incubators are exacerbated by a further issue, relating to the rate of O₂ diffusion into the cell culture media. During culture, cells adhere (or float in suspension) on a suitable substrate 1-2 cm below the liquid/gas interface between the media and gas space above, which acts effectively as a semi-permeable membrane. The time required for O₂ to diffuse out of cell culture media at 37° C. is minimally 80 minutes when a confluent monolayer of cells is present and maximally ≧180 minutes at low cell densities [11]. It can therefore take more than three hours for the O₂ concentration adjacent to the cells to equilibrate to the indicated incubator value. This means that following, for example, routine splitting and reseeding of cell cultures in fresh media, cells are exposed to undesirably high (initially atmospheric) concentrations of dissolved O₂ for a significant period, which may have important physiological effects.

Systems exist to allow transfer of culture materials from, for example, incubators to sealed glove boxes for manipulation without exposure to external atmospheric conditions (for example the C-Shuttle Glovebox, BioSpherix Ltd, Redfield N.Y., USA). In addition, attempts have been made to improve gaseous exchange in large scale cell culture apparatus, the use of aeration and agitation being generally known. Specialised techniques, such as the use of immiscible gas-bearing transfer liquids have also been attempted (WO 94/01530), although these have not, so far, been widely adopted.

Large-scale refrigerated storage under controlled atmospheres is known for perishable goods such as foodstuffs (GB 1,325,008, EP 0 517 046).

At its most general, the invention provides a method of preparing cell culture media for use in culturing cells by reducing the O₂ concentration in the cell culture media before culturing cells in the cell culture media. This may be achieved, for example, by exposing the cell culture media to a controlled atmosphere having a predetermined O₂ concentration that is less than an external atmospheric concentration of O₂.

By reducing the O₂ concentration in the cell culture media, cells which are subsequently added to the cell culture media can immediately be exposed to cell culture media having a reduced O₂ concentration. The cells can then be cultured in a cell culture incubator having a reduced O₂ concentration (e.g. 5%), without having to initially expose the cells to cell culture media having an undesirably high O₂ concentration. This has been found to result in improved cell growth.

In a first aspect, the invention provides a method as set out in claim 1.

By “external atmospheric O₂ concentration”, it is meant the normal or ambient atmospheric O₂ concentration that is found outside the first controlled atmosphere. Typically, this O₂ concentration is about 21%.

The first aspect of the invention therefore provides a method of preparing a cell culture media to have a reduced O₂ concentration, so that cells can subsequently be cultured in the cell culture media. In other words, the method allows a cell culture media to be prepared which has an O₂ concentration that is less than an O₂ concentration in the cell culture media when the cell culture media has been equilibrated with an external atmospheric O₂ concentration.

Once the method has been performed, cells which are subsequently added to the cell culture media can be immediately exposed to cell culture media having a reduced O₂ concentration. This can avoid exposing the cells to cell culture media having an undesirably high O₂ concentration. This has been found to result in improved cell growth.

The O₂ concentration in the cell culture media before exposing the cell culture to the first controlled atmosphere (i.e. an initial O₂ concentration) may be the O₂ concentration in the cell culture media after the cell culture media has been equilibrated with an external atmospheric O₂ concentration (which typically has a concentration of 21%). Typically, the O₂ concentration in aqueous mammalian cell culture media that has been equilibrated with an external atmospheric O₂ concentration is in the range 8% to 15%. Therefore, the O₂ concentration in the cell culture media before exposing the cell culture media to the first controlled atmosphere may be 4% or more, 6% or more, 8% or more, or 10% or more. The O₂ concentration in the cell culture media before exposing the cell culture media to the first controlled atmosphere may be 16% or less, or 14% or less.

The O₂ concentration in the cell culture media after exposing the cell culture media to the first controlled atmosphere (i.e. the resulting O₂ concentration) may be 10% or less, 8% or less, 6% or less, 5% or less, 4% or less, 3% or less, or 2% or less. It may be 0.1% or more, or 1% or more. An O₂ concentration in the cell culture media of about 2% (e.g. 1.5% to 2.5%) has been found to be particularly suitable for culturing cells.

The first controlled atmosphere may have a predetermined O₂ concentration of 15% or less, 10% or less, 5% or less, or 2% or less. It may be 0.1% or more. The first controlled atmosphere may be selected to achieve a predetermined O₂ concentration in the cell culture media. A first controlled atmosphere with an O₂ concentration of about 2% (e.g. 1.5% to 2.5%) has been found to be particularly suitable to produce cell culture media in which the O₂ concentration is about 2%.

The method may include exposing the cell culture media to the first controlled atmosphere for at least 1 minute, 1 hour, 3 hours, 6 hours, 12 hours, or 24 hours. The cell culture media may be exposed to the first controlled atmosphere for 72 hours or less, 48 hours or less, or 24 hours or less.

The method may include agitating the cell culture media in the first controlled atmosphere. It has been found that agitating the cell culture media can lead to a greater reduction in the O₂ concentration of cell culture media when exposed to an atmosphere having a lower O₂ concentration than an external atmospheric O₂ concentration. Agitating the cell culture media can also or alternatively help to increase the rate of reduction in the O₂ concentration of cell culture media when exposed to an atmosphere having a lower O₂ concentration than an external atmospheric O₂ concentration. Therefore, agitating the cell culture media can reduce the amount of time required for the O₂ concentration in cell culture media to reduce to a predetermined concentration (e.g. 2%).

The cell culture media may be agitated by vibration, rotation, sonication (applying sound to the cell culture media) and/or stirring of the cell culture media. The sound applied to the cell culture media may be ultrasound, e.g. sound having a frequency of 20 kHz or more.

The cell culture media may be agitated over a period of at least 1 minute, 1 hour, 3 hours, 6 hours, 12 hours, or 24 hours. The cell culture media may be agitated over a period of 72 hours or less, 48 hours or less, or 24 hours or less. The cell culture media may, for example, be intermittently, periodically or continuously agitated over this period.

The first controlled atmosphere may have a predetermined temperature. The first controlled atmosphere may have a predetermined temperature which is less than the temperature of a mammalian cell culture incubator (typically about 37° C.). The first controlled atmosphere may have a predetermined temperature which is 30° C. or less, or 25° C. or less. It may be around room temperature, e.g. 18° C. to 25° C. It has been found that having a temperature which is lower than 37° C. can lead to a greater reduction and/or rate of reduction in the O₂ concentration of cell culture media.

The first controlled atmosphere may have a predetermined temperature which is less than an external atmospheric temperature. Therefore, the method may include cooling the first controlled atmosphere below and external atmospheric temperature, e.g. by a refrigeration unit. By “external atmospheric temperature” it is meant the normal or ambient temperature external to the first controlled atmosphere. This temperature can be referred to as “room temperature” and is typically in the range 18° C. to 25° C. The first controlled atmosphere may therefore have a predetermined temperature which 20° C. or less, 15° C. or less, 10° C. or less, 5° C. or less, or about 4° C. (e.g. 3.5° C. to 4.5° C.). The first controlled atmosphere may have a predetermined temperature which is above 0° C.

It has been found that a first controlled atmosphere that has a predetermined temperature which is less than an external atmospheric temperature can help to increase the reduction and/or the rate of reduction in the O₂ concentration of cell culture media.

The first controlled atmosphere may have a predetermined pressure which is less than an external atmospheric pressure. Therefore, the pressure of the first controlled atmosphere may be reduced, e.g. by a pressure control unit. By “external atmospheric pressure” it is meant the normal or ambient pressure external to the first controlled atmosphere. At sea level, this pressure is typically 100 kPa. The predetermined pressure of the first controlled atmosphere may be at least 20 kPa, at least 40 kPa, or at least 70 kPa below an external atmospheric pressure. The predetermined pressure may be within a range bounded by any of these values. It is proposed that a reduction in pressure could help to increase the reduction and/or the rate of reduction in the O₂ concentration of cell culture media.

The first controlled atmosphere may have a predetermined pressure which is greater than an external atmospheric pressure. Therefore, the pressure of the first controlled atmosphere may be increased, e.g. by a pressure control unit. By “external atmospheric pressure” it is meant the normal or ambient pressure external to the first controlled atmosphere. At sea level, this pressure is typically 100 kPa. The predetermined pressure of the first controlled atmosphere may be at least 120 kPa, at least 140 kPa, or 170 kPa above an external atmospheric pressure. The predetermined pressure may be within a range bounded by any of these values. It is proposed that an increase in pressure could help to increase the reduction and/or the rate of reduction in the O₂ concentration of cell culture media.

The first controlled atmosphere may have a predetermined CO₂ concentration which is more than an external atmospheric CO₂ concentration. By “external atmospheric CO₂ concentration”, it is meant the normal or ambient atmospheric concentration of CO₂ that is found outside the first controlled atmosphere. Typically, this concentration of CO₂ is about 0.036%. The predetermined CO₂ concentration of the first controlled atmosphere may be 2% or more, it may be 5% or more, it may be about 5% (e.g. 4.5% to 5.5%). The predetermined CO₂ concentration of the first controlled atmosphere may be 10% or less. Increasing the CO₂ concentration in the first controlled atmosphere can increase the CO₂ concentration in the cell culture media, which can lead to enhanced cell growth when the cell culture media is subsequently used for culturing cells.

The cell culture media may be an aqueous cell culture media. The cell culture media may be a mammalian cell culture media. The method has been found to be particularly applicable to aqueous mammalian cell culture media. However, the method could equally be applied to other types of cell culture media.

The cell culture media may contain any one or more of salt (e.g. NaCl), nutrients, sugars and growth factors. The cell culture media may contain 4-8 grams per litre or 6-8 grams per litre of salt. As explained below, it is thought that the salt content of cell culture media may have an effect on how the O₂ concentration in cell culture media varies with temperature when exposed to an atmosphere having an O₂ concentration that is less than an external atmospheric O₂ concentration.

The cell culture media is preferably media that has not previously been used for cell culturing, e.g. it may be fresh and/or sterile.

The method may include sealing the vessel containing the cell culture media after or during exposing the cell culture media to the first controlled atmosphere. By sealing the vessel, the O₂ concentration in the cell culture media can be maintained at a reduced O₂ concentration resulting from exposing the cell culture media to the first controlled atmosphere. Thus sealed, the vessel may be stored (e.g. in an external atmospheric O₂ concentration) so that the cell culture media having a reduced O₂ concentration can be used for culturing cells at a later date.

The vessel may be sealed in the in the first controlled atmosphere, e.g. so that a “headspace” (space above the cell culture media) has the same gas concentration as the first controlled atmosphere. Alternatively, the vessel may be sealed after removing the vessel from the first controlled atmosphere. If so, the exposure of the cell culture media to an external atmosphere is preferably minimised so that the O₂ concentration in the cell culture media does not return to its original concentration.

The vessel may include a gas permeable portion, e.g. a gas permeable lid. A gas permeable portion is advantageous as it allows the cell culture media contained in the vessel to be exposed to the first controlled atmosphere, e.g. without having to remove a lid of the vessel. The gas permeable portion may be perforated and/or filtered, e.g. a perforated and/or filtered lid. The gas permeable portion may be sealable, so that the vessel can be sealed. For example, there may be provided a film for sealing the gas-permeable portion. Alternatively, there may be provided a gas permeable lid and a non gas permeable lid, so that the non gas permeable lid can be used to seal the vessel after exposure to the first controlled atmosphere through the gas permeable lid.

The method may include culturing cells in the cell culture media in a second controlled atmosphere, after the cell culture media has been exposed to the first controlled atmosphere. The second controlled atmosphere preferably has an O₂ concentration that is less than an external atmospheric O₂ concentration. It has been found that culturing cells by this method can result in improved cell growth when compared with cells cultured in cell culture media that did not have a reduced O₂ concentration before culturing cells in the media.

The second controlled atmosphere may be any atmosphere suitable for culturing cells. Preferably, the second controlled atmosphere has a predetermined O₂ concentration which is 10% or less, 5% or less, 2% or less. It may be 0.1% or more. The second controlled atmosphere may have a predetermined CO₂ concentration which is 2% or more, it may be 5% or more, it may be around 5% (e.g. 4.5% to 5.5%), it may be 10% or less. A second controlled atmosphere having a predetermined O₂ concentration of about 2% (e.g. 1.5% to 2.5%) and a predetermined CO₂ concentration of about 5% (e.g. 4.5% to 5.5%) has been found to be particularly suitable for culturing cells. The second controlled atmosphere preferably has a predetermined temperature of about 37° C. (e.g. 35° C. to 38° C. or 36.5° C. to 37.5° C.).

The first atmosphere may be the same as the second atmosphere. However, the first and second atmospheres are preferably different so that the first atmosphere can be optimised for preparing the cell culture media (e.g. to have reduced O₂ concentration) whereas the second controlled atmosphere can be optimised for cell growth.

The first and/or second controlled atmosphere may be provided in the same apparatus, e.g. a cell culture media preparation apparatus as described herein. Alternatively, the first controlled apparatus may be provided in a first apparatus (e.g. a cell culture media preparation apparatus as described herein) and the second controlled atmosphere may be provided in a second apparatus (e.g. a cell culture incubator).

In a second aspect, the invention provides a cell culture media preparation apparatus. The apparatus is preferably for carrying out the methods described herein. Accordingly, there may be provided a cell culture media preparation apparatus having:

a sealable chamber for holding one or more vessels containing cell culture media; and

an atmosphere control unit for providing a first controlled atmosphere within the chamber, the first controlled atmosphere having a predetermined O₂ concentration that is less than an external atmospheric O₂ concentration.

By sealing the chamber of the apparatus, a first controlled atmosphere (e.g. as described above) can be provided in the chamber. Therefore, the cell preparation apparatus is for carrying out the methods described in connection with the first aspect.

The atmosphere control unit may include an inert gas inlet for supplying an inert gas to the chamber therethrough. The atmosphere control unit may include an inert gas source for supplying an inert gas to the chamber, e.g. through the inert gas inlet. The inert gas may be Nitrogen (N₂). The first controlled atmosphere having a predetermined O₂ concentration may be provided in the chamber by supplying an inert gas to the chamber, e.g. to flush out the O₂ from the chamber.

The atmosphere control unit may include a CO₂ inlet for supplying CO₂ to the chamber therethrough. The atmosphere control unit may include a CO₂ source for supplying CO₂ to the chamber, e.g. through the CO₂ gas inlet. The first controlled atmosphere having a predetermined CO₂ concentration may be provided in the chamber by supplying CO₂ to the chamber.

The apparatus may include an agitation unit for agitating cell culture media in the first controlled atmosphere, e.g. by agitating the vessels of cell culture media. The agitation unit may be arranged to agitate cell culture media by vibration, rotation, sonication (applying sound to the cell culture media) and/or stirring of the cell culture media. Therefore, the agitation unit may include a vibrating platform (i.e. a platform arranged to be to be vibrated), a sound source and/or a stirring unit. The vibrating platform may be a platform supportable by legs which are arranged to vibrate the platform. The vibrating platform may be arranged to vibrate a vessel containing cell culture media so as to cause the cell culture media to agitate within the vessel. Such vibrating platforms are known.

The atmosphere control unit may include a temperature control unit for controlling the temperature of the first controlled atmosphere. The temperature control unit preferably includes a refrigeration unit for reducing the temperature of the first controlled atmosphere. The temperature control unit may include a heating unit for increasing the temperature of the first controlled atmosphere.

The atmosphere control unit may include a pressure control unit for controlling the pressure of the first controlled atmosphere. The pressure control unit preferably includes a vacuum pump for reducing the pressure of the first controlled atmosphere. The pressure control unit may include a pump for increasing the pressure of the first controlled atmosphere.

The atmosphere control unit may include one or more sensors for sensing or detecting one or more parameters of the first controlled atmosphere. The parameters may include O₂ concentration, CO₂ concentration, temperature and/or pressure. For example, the atmosphere control unit may include any one or more of: an O₂ sensor for sensing an O₂ concentration of the first controlled atmosphere; a CO₂ sensor for sensing a CO₂ concentration of the first controlled atmosphere; a temperature sensor for sensing a temperature of the first controlled atmosphere; and a pressure sensor for sensing a pressure of the first controlled atmosphere.

The atmosphere control unit may include a controller for controlling the first controlled atmosphere. The controller may control the first controlled atmosphere according to predetermined parameters, e.g. a predetermined O₂ concentration, CO₂ concentration, temperature and/or pressure of the first controlled atmosphere. The controller may be operable by a user to set the predetermined parameters. Alternatively, the predetermined parameters may be preset (i.e. so that input from a user is not required).

The controller may be arranged to control any one or more of: O₂ concentration, CO₂ concentration, temperature and/or pressure of the first controlled atmosphere. To achieve this, the controller may be arranged to control any one or more of the inert gas source, the CO₂ source, the temperature control unit and the pressure control unit described above.

The controller may further be connected to any one or more of the sensors described above, to allow the controller to monitor one or more parameters of the first controlled atmosphere. The controller may be connected to a display for displaying one or more parameters of the first controlled atmosphere.

The agitation unit (if present) may be operable by a user to control the agitation unit. The controller may be arranged to control the agitation unit.

The chamber of the apparatus may be sterilisable and/or sterile. This reduces the likelihood of cell culture media in the chamber being contaminated.

The chamber may contain a first controlled atmosphere having a predetermined O₂ concentration that is less than an external atmospheric O₂ concentration. The first controlled atmosphere may have a predetermined CO₂ concentration, temperature and/or pressure. The O₂ concentration, CO₂ concentration, temperature and/or pressure of the first controlled atmosphere may be as described in connection with the first aspect above.

The apparatus may contain one or more vessels of cell culture media. The cell culture media may be aqueous cell culture media. It may be mammalian cell culture media. The vessels may include a gas permeable portion, e.g. a gas permeable lid. A gas permeable portion is advantageous as it allows the cell culture media contained in the vessel to be exposed to the first controlled atmosphere, e.g. without having to remove a lid of the vessel. The gas permeable portion may be sealable, so that the vessel can be sealed. For example, there may be provided a film for sealing the gas-permeable portion. Alternatively, there may be provided a gas permeable lid and a non gas permeable lid, so that the non gas permeable lid can be used to seal the vessel after exposure to the first controlled atmosphere through the gas permeable lid.

In a third aspect, the invention provides a method of cell culture which includes:

providing a vessel containing cell culture media, the cell culture media having an O₂ concentration which is less than an O₂ concentration in the cell culture media when the cell

culture media has been equilibrated with an external atmospheric O₂ concentration; and culturing cells in the cell culture media in a second controlled atmosphere having an O₂ concentration which is less than an external atmospheric O₂ concentration.

Providing cell culture media having a reduced O₂ concentration (i.e. a concentration which is below an equilibrium O₂ concentration) before culturing cells in the cell culture media can avoid exposing the cells to cell culture media having an undesirably high O₂ concentration. As explained previously, this has been found to result in improved cell growth.

The cell culture media is preferably prepared according to the method of the first aspect, but this is not required since it is the reduced O₂ content of the cell culture media which is thought to be responsible for improved cell growth.

The cell culture media may have any feature of a cell culture media prepared according to the method of the first aspect. For example, the O₂ concentration in the cell culture media before culturing cells in the cell culture media may be 10% or less, 8% or less, 6% or less, 5% or less, 4% or less, 3% or less, or 2% or less. Preferably, the O₂ concentration in the cell culture media before culturing cells in the cell culture media is 0.1% or more. An O₂ concentration in the cell culture media of about 2% (e.g. 1.5% to 2.5%) has been found to be particularly suitable for culturing cells.

The second controlled atmosphere in which the cells are cultured may be any atmosphere suitable for culturing cells, particularly mammalian cells, provided it has a reduced O₂ concentration. The second controlled atmosphere may be the second controlled atmosphere described in connection with the first aspect.

In a fourth aspect, the invention provides a vessel containing cell culture media having an O₂ concentration which is less than an O₂ concentration in the cell culture media when the cell culture media has been equilibrated with an external atmospheric O₂ concentration.

For reasons already explained, such a cell culture media has been found to lead to improved cell growth when used in cell culturing. The vessel containing cell culture media is preferably prepared according to the methods described above, but this need not be the case.

The cell culture media may have any feature of a cell culture media prepared according to the method of the first aspect. For example, the O₂ concentration in the cell culture media may be 10% or less, 8% or less, 6% or less, 5% or less, 4% or less, 3% or less or 2% or less. Preferably, the O₂ concentration in the cell culture media is 0.1% or more. An O₂ concentration in the cell culture media of about 2% (e.g. 1.5% to 2.5%) has been found to be particularly suitable for culturing of cells.

The vessel is preferably sealed, to maintain its O₂ concentration. The vessel may include a sealed or sealable gas-permeable portion.

In a fifth aspect, the invention provides a refrigerated device for exposing containers of liquid media to a defined atmosphere, said device being sealable so as to provide an essentially gas-tight chamber, characterised in that the device is provided with means for supplying one or more gases and means for detecting the concentration of O₂, such that a predetermined concentration of O₂ may be maintained within the chamber.

Preferably, the one or more gases supplied are selected from the list consisting of N₂, CO₂, O₂ and an inert gas.

In a preferred embodiment, the O₂ within the chamber may be maintained at less than normal atmospheric concentration (21%), preferably at less than 15%, more preferably at less that 10%, still more preferably at less then 5%, most preferably at less than 3%. In a highly preferred embodiment, the concentration of O₂ is maintained at about 2%. In one preferred embodiment the device comprises a display providing a read-out of the internal concentration of O₂.

The device preferably further comprises a CO₂ detector and means for supplying CO₂, such that a predetermined concentration of CO₂ may be maintained within the chamber. In alternative embodiments, the device comprises a display further providing a readout of one or more of: temperature, concentration of CO₂, concentration of N₂, or relative humidity.

The device may comprise means by which the pressure of gas within the chamber may be reduced to below atmospheric pressure. Preferably this comprises a pump but alternatively the device may be connected to a vacuum supply. In this embodiment the device also preferably comprises a display providing a read-out of the pressure within the chamber.

The device may comprise means by which the pressure of gas within the chamber may be increased to above atmospheric pressure. Preferably this comprises a pump. In this embodiment the device also preferably comprises a display providing a read-out of the pressure within the chamber.

In a sixth aspect, the invention provides a method of cell culture comprising the step of pre-equilibrating cooled medium with oxygen at a concentration less than normal atmospheric concentration. Preferably, the medium is cooled to 10° C. or less before or during the pre-equilibration step. The method may optionally further comprise an initial step of degassing the medium by reducing the pressure within the chamber. This step may be repeated to further accelerate the rate of equilibration. Preferably, the pressure is reduced by at least 200 mbar below atmospheric pressure, more preferably at least 400 mbar below atmospheric pressure, most preferably at least 700 mbar below atmospheric pressure. Preferably the oxygen content of the medium is equilibrated with an atmosphere comprising less than 15% O₂, more preferably at less that 10%, still more preferably at less then 5%, most preferably at less than 3%. In a highly preferred embodiment, the concentration of O₂ is maintained at about 2%.

In this specification cultured cells may be of any kind, but are preferably mammalian cells. They may be non-human cells, e.g. rabbit, guinea pig, rat, mouse or other rodent (including cells from any animal in the order Rodentia), cat, dog, pig, sheep, goat, cattle, horse, non-human primate or other non-human vertebrate organism; and/or non-human mammalian cells; and/or human cells.

The cultured cells may be stem cells. The stem cells may be stem cells of any kind. They may be embryonic stem cells (ESC), e.g. human or non-human embryonic stem cells (hESC). Alternatively they may be adult stem cells, human or non-human.

In yet a further aspect of the present invention, a pharmaceutical composition comprising stem cells generated by any of the methods of the present invention, or fragments or products thereof, is provided. The pharmaceutical composition may be useful in a method of medical treatment. Suitable pharmaceutical compositions may further comprise a pharmaceutically acceptable carrier, adjuvant or diluent.

In another aspect of the present invention, stem cells generated by any of the methods of the present invention may be used in a method of medical treatment, preferably, a method of medical treatment is provided comprising administering to an individual in need of treatment a therapeutically effective amount of said medicament or pharmaceutical composition.

Stem cells obtained through culture methods and techniques according to this invention may be used to differentiate into another cell type for use in a method of medical treatment. Thus, the differentiated cell type may be derived from, and may be considered as a product of, a stem cell obtained by the culture methods and techniques described which has subsequently been permitted to differentiate. Pharmaceutical compositions may be provided comprising such differentiated cells, optionally together with a pharmaceutically acceptable carrier, adjuvant or diluent.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

The details of one or more embodiments of the invention are set forth in the accompanying description below including specific details of the best mode contemplated by the inventors for carrying out the invention, by way of example. It will be apparent to one skilled in the art that the present invention may be practiced without limitation to these specific details.

The present invention is concerned with the preparation of culture media of a type suitable for culture of mammalian cells. Typically, mammalian cell culture media has an oxygen concentration of about 8 to 15%. This is much higher than physiological oxygen concentrations in mammalian tissue. Although the culture of mammalian cells may be conducted in a surrounding atmosphere having a controlled oxygen concentration that is considerably reduced compared to the oxygen concentration of the normal atmosphere (about 21% oxygen), the inventors have observed that equilibration of oxygen in the culture media with the surrounding atmosphere takes considerable time meaning that the cells in culture remain exposed to relatively high oxygen concentrations for a considerable part of the culture time.

The inventors have observed that by altering the oxygen concentration of the culture media prior to addition of cells for culture can improve the yield of the cell culture. The inventors have also separately observed the effects of agitation of the culture media and cooling of the culture media on the rate of equilibration of the oxygen concentration of the culture media with that of the surrounding atmosphere.

A culture media surrounded by a gaseous atmosphere has a gas:media interface across which gases may be exchanged. The exchange of oxygen across this interface alters the oxygen concentration of the culture media. The inventors have now found that the rate of exchange of oxygen across this interface is separately affected by the temperature of the culture media and by agitation of the media.

Agitation of the culture media has been found to significantly increase the rate of oxygen exchange across the interface enabling the a reduction of the oxygen concentration of culture media to be achieved through incubation in a reduced oxygen atmosphere in much shorter times than was previously possible.

Reducing the temperature of the culture media below that used for culture of mammalian cells (typically about 35-38° C., more typically about 37° C.) has also been found to increase the rate at which oxygen may be exchanged across the interface. Accordingly, reduction of oxygen concentration in culture media incubated in a reduced oxygen atmosphere may also be achieved in much shorter times than previously possible by conducting the incubation at reduced temperatures, e.g. below about 30° C., more preferably below about 25° C., still more preferably below about 22° C., below about 20° C., below about 15° C., below about 10° C., below about 5° C. or about 4° C. (e.g. 3.5° C. to 4.5° C.).

The increase in rate of oxygen exchange across the interface at lower temperatures is unexpected given that Brownian motion and diffusion, which may be thought to affect exchange of molecules across an interface, would normally be considered to increase at higher temperatures. Whilst this application is not to be bound by any theory, one suggestion for the increase in rate of exchange of oxygen across the interface at lower temperatures is that the solubility of oxygen in salt-containing mammalian cell culture media is altered at lower temperatures such that more oxygen may cross the interface and escape the aqueous culture media.

These novel insights have led the inventors to propose a method of preparing culture media for mammalian cell culture by incubating culture media so as to reduce the oxygen concentration of the culture media prior to addition of cells to be cultured. In preferred embodiments the reduction of oxygen concentration in the culture media is achieved by incubating the culture media in a surrounding gaseous environment that has a controlled oxygen concentration that is less than the oxygen concentration of the culture media. Typically the culture media before incubation of the cell culture media is about 14% or less, 12% or less, or 10% or less. The surrounding gaseous environment preferably has an oxygen concentration of about 10% or less, more preferably about 8% or less, 6% or less, 4% or less, or 2% or less. It may be 0.1% or more. At the end of the treatment the oxygen concentration in the culture media may be one of about 10% or less, about 8% or less, about 6% or less, about 4% or less, about 3% or less or 2% or less.

In a preferred embodiment during incubation the culture media may be agitated in order to increase the rate of oxygen exchange across the interface.

In another preferred embodiment the culture media may be incubated at reduced temperature of 35° C. or less, more preferably 30° C. or less, still more preferably 25° C. or less in order to increase the rate of oxygen exchange across the interface. Temperatures of 20° C. or less, 15° C. or less, 10° C. or less, or 5° C. or less may be preferred.

Culture media prepared/treated in this way has a lower oxygen content compared to non-prepared/non-treated culture media. The inventors have found that prepared/treated culture media improves the yield of cells cultured in the media.

Embodiments and experiments illustrating the principles of the invention will now be discussed with reference to the accompanying figures in which:

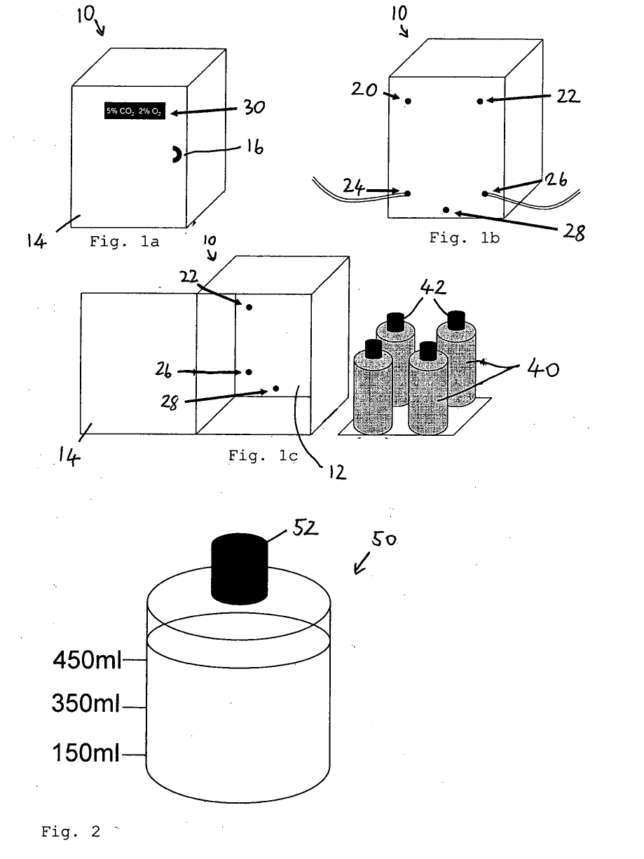

FIGS. 1a-c are drawings of a first cell culture media preparation apparatus.

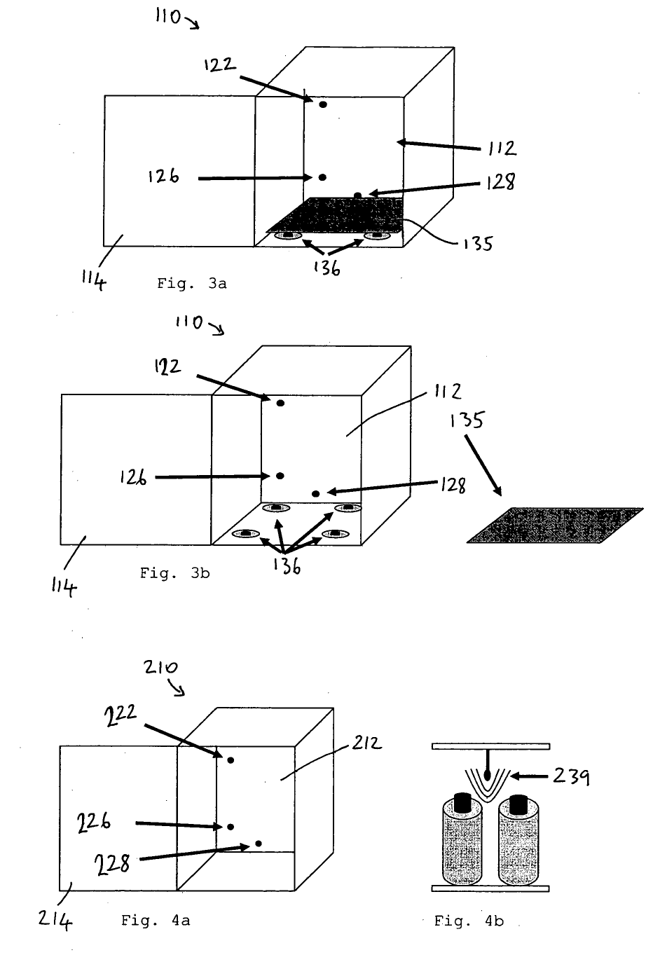

FIG. 2 is a perspective view of a bottle for containing cell culture media.

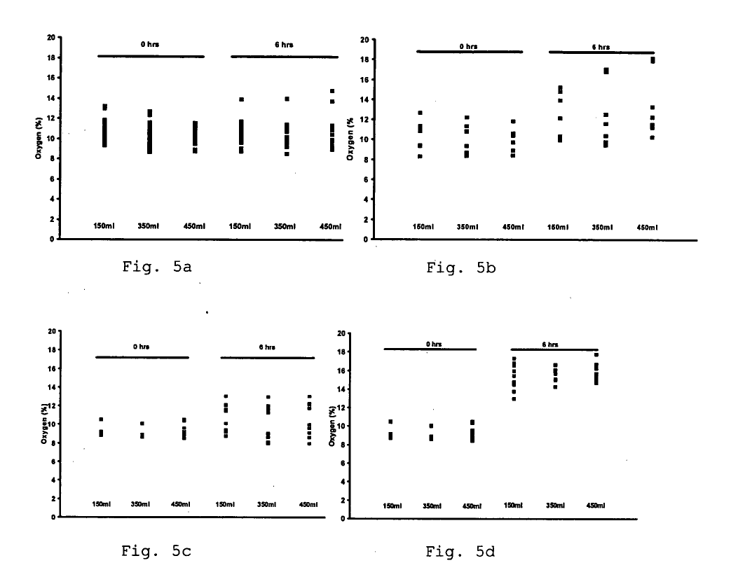

FIGS. 3a and 3b are drawings of a second cell culture media preparation apparatus.

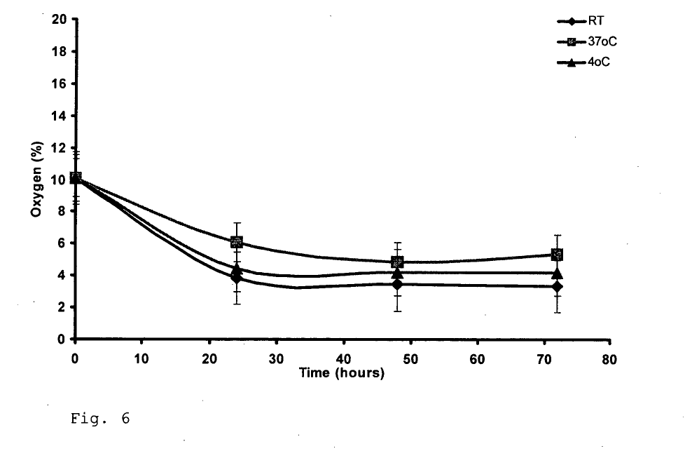

FIGS. 4a and 4b are drawings of a third cell culture media preparation apparatus.

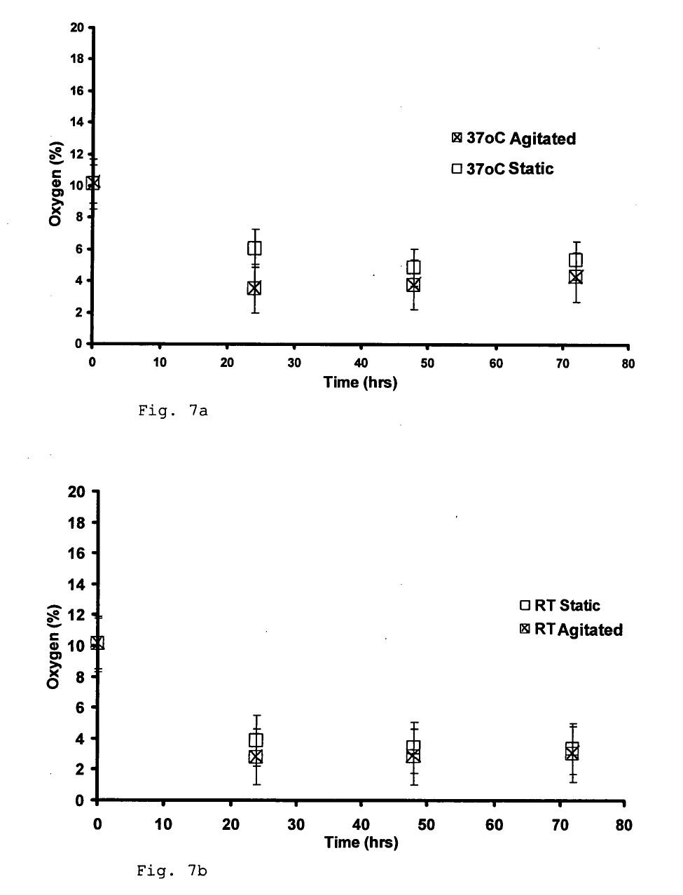

FIGS. 5a-d are graphs comparing O₂ concentration in cell culture media with time and depth in the cell culture media.

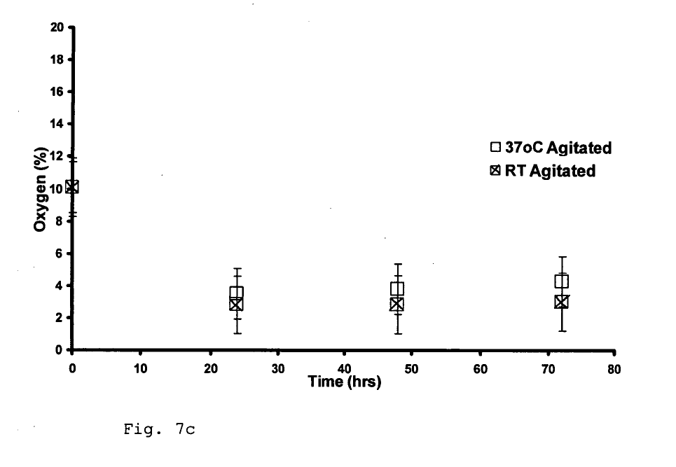

FIG. 6 is a graph comparing O₂ concentration in cell culture media with time.

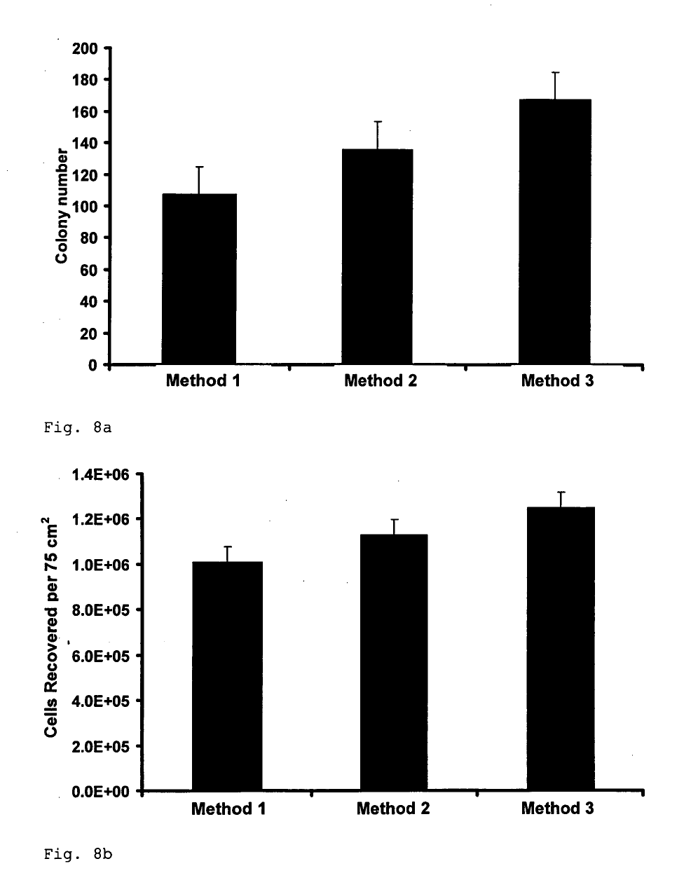

FIGS. 7a-c are graphs comparing O₂ concentration in cell culture media with time under different conditions.

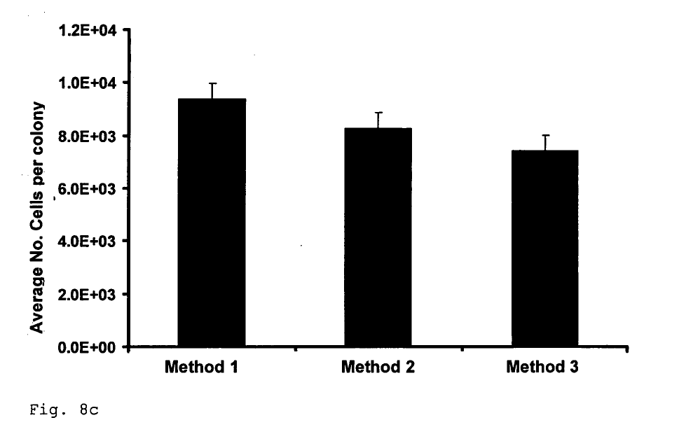

FIGS. 8a-c are graphs comparing colony units formed, cells recovered, and cells per colony for cells cultured according to different methods.

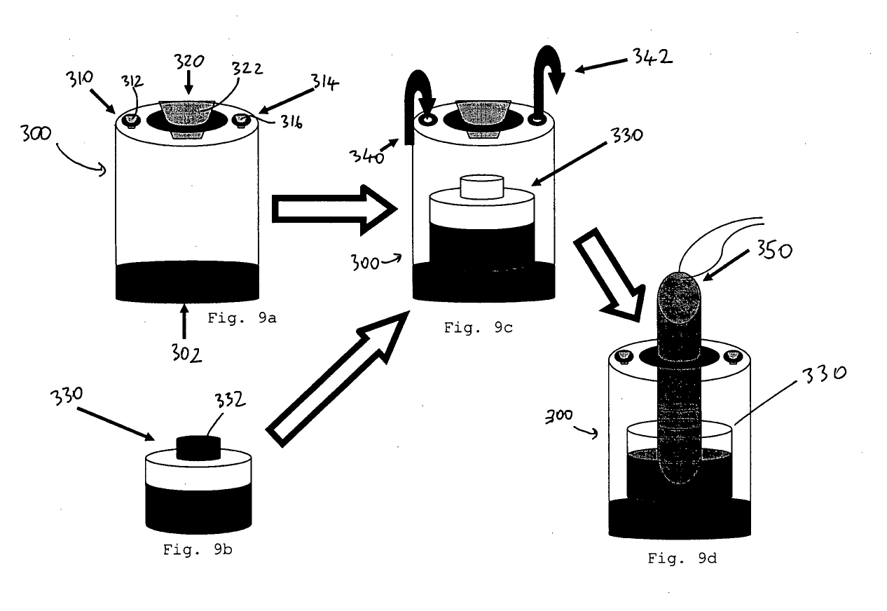

FIGS. 9a-d are drawings illustrating a method of measuring O₂ concentration in cell culture media.

FIG. 1a is a view of the front of a first cell culture media preparation apparatus 10. The apparatus 10 has an essentially cuboidal or rectangular shape which defines a chamber 12 (see FIG. 1c). The apparatus 10 has a door 14 which provides an essentially gastight seal to the chamber 12 when closed. A handle 16 allows the door 14 to be opened and closed.

The apparatus 10 includes an atmosphere control unit (not shown) for providing a first controlled atmosphere within the chamber 12.

FIG. 1b is a view of the back of the apparatus 10 showing exemplary arrangement of an O₂ sensor 20, a CO₂ sensor 22, an N₂ inlet 24 and a CO₂ inlet 26, which form part of the atmosphere control unit. The N₂ inlet 24 and CO₂ inlet 26 are connected to an N₂ source and a CO₂ source respectively (not shown). Optionally, a pressure release valve 28 is provided.

The atmosphere control unit includes a temperature control unit (not shown) which in this case is a refrigeration unit that includes refrigeration components such as a compressor and heat exchange coils to enable the temperature within the chamber 12 to be reduced below room temperature. In some embodiments, the temperature control unit includes a heater. The atmosphere control unit also includes a temperature sensor (not shown) for detecting the temperature within the chamber 12.

The atmosphere control unit may optionally include an integral pump, in this case a vacuum pump (not shown), to allow the pressure within the cavity to be controlled (e.g. reduced) below an external atmospheric pressure (which is usually about 100 kPa). In an alternative embodiment, a coupling for an external pump may be provided, e.g. where an integral pump is not provided. The atmosphere control unit may also include a pressure sensor (not shown) for detecting the pressure within the chamber 12.

FIG. 1c is a front view of the apparatus 10 with the door 14 open. The chamber 12 and internal apertures for the O₂ and CO₂ sensors 20, 22, N₂ and CO₂ gas inlets 24, 26 and the pressure release valve 28 are shown. Again, the refrigeration components such as the internal heat exchange coils are not shown. The chamber 12 may be of a suitable size to house a convenient number (e.g. four) of standard 500 ml or 1000 ml cell culture media bottles. As an example, the apparatus 10 may be 33 cm high, 40 cm wide and 40 cm deep.

In this embodiment, the apparatus 10 is provided with a display 30 to provide information as to the atmosphere within the chamber 12 of the apparatus 10, e.g. as detected by the sensors. The information provided by the display 30 may include temperature, O₂ concentration, CO₂ concentration, relative humidity, and/or internal pressure of the atmosphere within the chamber 12.

The atmosphere control unit includes a controller (not shown) for controlling the atmosphere within the chamber 12. The controller controls the atmosphere in the chamber 12 according to predetermined parameters. The predetermined parameters may, for example, include O₂ concentration, CO₂ concentration, temperature and/or pressure. In this embodiment, the controller is operable by a user to set predetermined parameters. In other embodiments, the parameters are preset. Some or all of the predetermined parameters may be displayed on the display 30.

The controller may be arranged to reduce the O₂ concentration of the atmosphere within the chamber 12 by instructing the N₂ source to supply N₂ to the chamber 12 (via the N₂ inlet 24). The controller may be arranged to increase the CO₂ concentration of the atmosphere within the chamber 12 by instructing the CO₂ source to supply CO₂ (via the CO₂ inlet 26). The controller may be arranged to control the temperature within the chamber 12 by instructing the temperature control unit (e.g. the refrigeration unit) to operate. The controller may be arranged to reduce/increase the pressure in the chamber by instructing an integral/external pump to operate (if present).

The controller may use information from the sensors (e.g. the O₂, CO₂, temperature and/or pressure sensors) to control the atmosphere according to the predetermined parameters described above. For example, the controller may be arranged to control the temperature within the chamber to be a predetermined temperature by operating a refrigeration unit if the temperature is above a predetermined temperature. The O₂ concentration, CO₂ concentration, and pressure can be controlled in a similar manner.

FIG. 1c shows four 1000 ml bottles 40 which each contain 1000 ml of cell culture media. The bottles 40 have lids 42 which are gas-permeable, e.g. perforated and/or filtered. Preferably the lids 42 are of a suitable design so as to maintain sterility of cell culture medium, while allowing gas diffusion, e.g. by having an appropriate pore size.

In order to reduce the O₂ concentration in the cell culture media contained in the bottles 40, the bottles 40 are placed in the chamber 12 of the apparatus 10. The door 12 is then closed to provide a gastight seal to the chamber 12.

The control unit then controls the atmosphere within the chamber 12 according to predetermined parameters. In this case, the predetermined parameters include an O₂ concentration of 2% and a CO₂ concentration of 5% (as shown on the display 30). The controlled atmosphere within the chamber 12 diffuses through the gas-permeable lids 42 and therefore into the headspace of the bottles 40, therefore exposing the cell culture media within each bottle 40 to the controlled atmosphere. Because the controlled atmosphere has a low (2%) O₂ concentration (which is significantly lower than external atmospheric concentrations), the O₂ concentration in the cell culture media is reduced.

The gas permeable lids 42 are sealable so that the bottles 40 can be sealed when they are removed from the chamber 12. This means that the cell culture media in the bottles can be prevented from being exposed to external atmospheric concentrations of gasses (e.g. an O₂ concentration of 21%) when removed from the chamber 12. In this example, the lids 42 are sealed by applying a gas-tight film placed across a gas permeable membrane of the lids 42.

The apparatus 10 may further include an agitation unit for agitating cell culture media in the first controlled atmosphere within the chamber 12. The agitation unit may be operable by a user to control the agitation unit. In different embodiments, the agitation unit may include a vibrating platform, a sound source and/or a stirring unit.

As explained above, the O₂ concentration in the cell culture media can be reduced simply by exposing the cell culture media to an O₂ concentration which is less than external atmospheric concentrations of O₂ (typically 21%). However, it has been found that agitating the cell culture media during exposure to the first controlled atmosphere can lead to an increase in the reduction and/or the rate of reduction in the O₂ concentration of cell culture media. Agitation may be achieved using the agitation unit described above.

It has been found that reducing the temperature of the controlled atmosphere in the chamber 12 below that of an external atmospheric temperature (e.g. room temperature) can lead to an increase in the reduction and/or the rate of reduction in the O₂ concentration of cell culture media. A reduced temperature within the chamber 12 can be achieved using the atmosphere control unit (as described above).

It is proposed that increasing/reducing the pressure of the controlled atmosphere in the chamber 12 above/below that of an external atmospheric pressure (e.g. sea level pressure which is approximately 100 kPa) can lead to an increase in the reduction and/or the rate of reduction in the O₂ concentration of cell culture media. An increased/reduced pressure within the chamber 12 can be achieved using an integral/external pump and the control unit (as described above).

It may be desirable to generate standard curves for predicting the time required for cell culture media contained in particular sizes of bottles to reach a desired gas concentration (e.g. of O₂) at given conditions of temperature and pressure.

FIG. 2 is a perspective view of a 500 ml bottle 50 for containing cell culture media which may be used instead of the 1000 ml bottles shown in FIG. 1. As with the bottles 40, each bottle 50 includes a gas-permeable lid 52. FIG. 2 includes measurement markings to indicate the depths at which the bottle 50 is filled to volumes of 150 ml, 350 ml and 450 ml.

FIGS. 3a and 3b are views of the front of a second cell culture media preparation apparatus 110. Features in common with the first cell culture media preparation apparatus 10 are labelled with corresponding reference numbers and shall not be explained in further detail.

The apparatus 110 includes a platform (or “stage”) 135 supported by four legs 136. FIG. 3b shows the apparatus 110 with the platform 135 removed. The legs 136 are arranged to vibrate the platform 135 so as to cause the cell culture media within vessels of cell culture media located on the platform to agitate. The platform 135 may be smaller than the size of the chamber 12 to allow vibration of the platform 135 within the plane of the platform.

FIG. 4a is a view of the front of a third cell culture media preparation apparatus 210. Features in common with the first cell culture media preparation apparatus 10 are labelled with corresponding reference numbers and shall not be explained in further detail.

The apparatus 210 includes a sound source 239 for agitating cell culture media by applying sound thereto. FIG. 4b shows the sound source 239 in more detail. In this embodiment the sound source 239 is a source of ultrasound.

Although water is a liquid, it usually contains a significant amount of dissolved oxygen plus small amounts of other gasses. Icy cold water (0° C.) can hold as much as 4.9% oxygen by volume. However, as the water's temperature increases its ability to hold oxygen decreases. Table 1 below lists the maximum amount of oxygen that can be dissolved in water at different temperatures.

TABLE 1

| Temperature | Oxygen |
|---|---|
| (° C.) | (Max % by volume) |
| 0 | 4.9 |
| 10 | 3.8 |
| 20 | 3.1 |
| 30 | 2.6 |

Measurements have shown that temperature has a much greater impact on the oxygen diffusion coefficient than the gas mixture composition, with O₂ concentration of secondary importance and relative humidity having little effect [12]. However, it is important to remember that the oxygen concentration enters the diffusion equation in two ways, both embedded in the diffusion coefficient (through the O₂ diffusion coefficient as described above), and as the driving force through its concentration gradient. As the driving force, the O₂ concentration gradient does have a very significant effect on the magnitude of diffusive oxygen transport [12].

Measurements have also shown that the diffusion coefficient of oxygen in water is approximately 5 orders of magnitude less than seen in air [13]. It has also been shown that with increasing temperature the viscosity of water decreases and is accompanied by an increased oxygen diffusion coefficient [13].

Experiments illustrating the principles of the invention will now be discussed with reference to Examples 1 to 4.

In all experiments, O₂ concentrations of cell culture media were measured with the Dissolved Oxygen Bench Meter (Model No HI2400, Hanna Instruments). The instrument was calibrated at 0% O₂ using Zero Oxygen Solution (Part No H17040, Hanna Instruments) and at 21% O₂ using the external atmosphere prior to recording measurements. To maintain experimental control Dulbeccos Modified Eagles Media (DMEM) was used as the cell culture media throughout which was sourced from Lonza (Cat no 12-709F).

In all experiments, agitation of cell culture media was achieved by placing vessels containing the cell culture media on a vibrating platform which caused cell culture media contained within the vessels to rotate at a constant rotational speed of 2 rotations/per second using a generic device.

### EXAMPLE 1

FIGS. 5a-d show the results of an experiment to test the effect of agitation and temperature on the O₂ concentration in cell culture media exposed to normal external atmospheric concentrations of O₂ (21%).

The results of FIG. 5a-d were obtained by providing a 500 ml bottle of cell culture media (similar to the bottle 50 shown in FIG. 2) and exposing the cell culture media inside the bottle to the external atmosphere. In this experiment, the bottles used did not have gas permeable lids and so the cell culture media was exposed to the external atmosphere by removing the lid. Measurements of O₂ concentrations in the cell culture media were taken at depths corresponding to the bottle being filled to volumes of 150 ml, 350 ml and 450 ml.

FIG. 5a shows the results of measurements taken for room temperature cell culture media in normal atmospheric conditions (21% O₂ concentration, room temperature) after 0 hours and 6 hours. As can be seen from FIG. 5a, the O₂ concentration in the cell culture media is in the range 8-15%. No significant changes (p≦0.05) in O₂ concentration were noted after the 6 hours exposure to normal atmospheric conditions (21% O₂ concentration, room temperature).

FIG. 5b shows the results for measurements taken for room temperature cell culture media undergoing agitation in normal atmospheric conditions (21% O₂ concentration, room temperature) after 0 hours and 6 hours. As can be seen from FIG. 5b, agitating the cell culture media causes the O₂ concentration in the cell culture media to increase towards the external atmospheric concentration of 21%. A solitary significant change (p=0.031) in O₂ concentration was noted after 6 hours exposure to normal atmospheric conditions (21% O₂ concentration, room temperature) with accompanying agitation. This significant change occurred at the 450 ml measuring position (FIG. 2) which is adjacent to the gas liquid interphase.

FIG. 5c shows the results for measurements taken for 4° C. pre-chilled cell culture media in a controlled atmosphere having a temperature of 4° C. and an external atmospheric concentration of O₂ (21%). As can be seen from FIG. 5c, reducing the temperature of the cell culture media causes the O₂ concentration in the cell culture media to increase towards the external atmospheric concentration (21%). However, no significant changes (p≦0.05) in O₂ concentration were noted after the 6 hours exposure to the controlled atmosphere (21% O₂ concentration, 4° C.).

FIG. 5d shows the results for measurements taken for 4° C. pre-chilled cell culture media undergoing agitation in a controlled atmosphere having a temperature of 4° C. and an external atmospheric concentration of O₂ (21%). As can be seen from FIG. 5d, both agitating the cell culture media and reducing the temperature of the cell culture media cause the O₂ concentration in the cell culture media to increase towards the external atmospheric concentration of 21%. Significant changes in O₂ concentration were noted after 6 hours of exposure to the controlled atmosphere (21% O₂ concentration, 4° C.) with accompanying agitation. These significant changes occurred at all measuring positions; 450 ml (p=3.71E-08), 350 ml (p=3.85E-09), and 150 ml (p=7.06496E-07).

FIGS. 5b and 5d suggest that agitating cell culture media encourages the O₂ concentration in the cell culture media to move towards the O₂ concentration of the atmosphere to which it is exposed.

FIGS. 5c and 5d suggest that reducing the temperature of cell culture media encourages the O₂ concentration in the cell culture media to move towards the O₂ concentration of the atmosphere to which it is exposed.

### EXAMPLE 2

FIG. 6 shows the results of an experiment to test the effect of exposing cell culture media to a controlled atmosphere having an O₂ concentration of 2%. The method used for the experiment is shown in FIGS. 9a to 9d.

The experiment involved using a 1 litre screw top polypropylene jar 300 having a removable base 302 (see FIG. 9a) and a cell culture media bottle 330 containing 150 ml of cell culture media (see FIG. 9b). In the top of the jar 300 is a gas inlet 310 which is plugged by a stopper 312, a gas outlet 314 which is plugged by a stopper 316 and a meter inlet 320 which is plugged by a stopper 322. The cell culture media bottle 330 includes a non gas permeable lid 332.

The experiment involved removing the lid 332 of the bottle 330 and then placing the bottle 330 in the jar 300 by removing and then replacing the lid 302 to seal the jar 300. The gas inlet stopper 312 and gas outlet stopper 316 were then removed and gas having a 2% O₂ concentration was provided through the gas inlet 310 as indicated by arrow 340 (see FIG. 9c). The gas having a 2% O₂ concentration caused the displacement of gas having an external atmospheric O₂ concentration (21%) from the jar 300, as indicated by arrow 342 (see. FIG. 9c). The gas inlet 310 and gas outlet 314 were then re-plugged by the stoppers 312, 316 to maintain a controlled atmosphere having a 2% O₂ concentration within the jar 300.

O₂ concentration in the cell culture media was measured using a meter 350 after 24 hours, 48 hours and 72 hours. To make these measurements, the meter inlet stopper 322 was removed and a dissolved O₂ meter 350 inserted in the meter inlet 320 (see FIG. 9d). Measurements were recorded using the meter 350 once readings from the meter 350 had stabilised. After taking the measurements, the meter inlet 320 was re-plugged by the meter inlet stopper 322 to reseal the jar 300.

The experiment was carried out at temperatures within the jar 300 of 4° C., room temperature and 37° C.

FIG. 6 suggests that exposing cell culture media to a controlled atmosphere having a reduced O₂ concentration (in this case 2%) causes the O₂ concentration of the cell culture media to reduce.

In addition, FIG. 6 suggests that there is an increase in the reduction and/or rate of reduction in the O₂ concentration of cell culture media when the cell culture media is exposed to a temperature of 4° C. (p=0.003434) and room temperature (p=0.005135), when compared with a (higher) temperature of 37° C. In other words, FIG. 6 suggests that a lower temperature can provide an increased reduction and/or rate of reduction in the O₂ concentration of cell culture media.

The results of FIG. 6 are surprising when considered alongside Table 1 which suggests that reducing the temperature of water reduces its ability to hold O₂. The results of FIG. 6 are also surprising when considered alongside previous measurements showing that increasing temperature increases the oxygen diffusion coefficient of water [13]. Although the inventors do not wish to be bound by theory, a possible explanation for the result shown in FIG. 6 may be the salt content of cell culture media having an effect on the way in which O₂ dissolves in and/or diffuses out of cell culture media.

FIG. 6 shows that the reduction in O₂ concentration in cell culture media was slightly greater when the cell culture media was at room temperature than it was when the cell culture media was at 4° C., when it might be expected that the reduction in O₂ concentration in cell culture media would be greater for a temperature of 4° C. It is suggested that this possible anomaly may have been caused by the way in which the O₂ concentrations were measured, since O₂ concentration measurements were taken whilst temporarily removing the stopper 322, thus allowing some gas having normal atmospheric O₂ concentrations into the jar 300.

### EXAMPLE 3

FIGS. 7a-c show the results of an experiment to test the effect of agitating cell culture media when it is exposed to a controlled atmosphere having a 2% O₂ concentration.

The method used for this experiment is the same as that described with reference to FIG. 9 under Example 2 above, except that the experiment was carried out for cell culture which was agitated and cell culture that was static (not agitated) at temperatures within the jar 300 of room temperature and 37° C.

FIG. 7a show a comparison of O₂ concentration in static/agitated cell culture media at 37° C. FIG. 7b shows a comparison of O₂ concentration in static/agitated cell culture media at room temperature. In both cases, there is a significant reduction in the O₂ concentration in the cell culture media. This reduction was significantly greater when the cell culture media was agitated; room temperature (p=0.000125) and 37° C. (p=0.000254). This suggests that agitating cell culture media can increase the reduction and/or rate of reduction in the O₂ concentration of cell culture media when exposed to an atmosphere having a low O₂ concentration.

FIG. 7c shows a comparison of O₂ concentration in agitated cell culture media at room temperature and at 37° C. FIG. 5c shows that the reduction in O₂ concentration in the cell culture media was greater at room temperature then it was for the higher temperature of 37° C. (p=0.027543). FIG. 5c therefore suggests that a lower temperature can increase the reduction and/or rate of reduction in the O₂ concentration of cell culture media when exposed to an atmosphere having a low O₂ concentration.

### EXAMPLE 4

FIGS. 8a-c show the results of an experiment to compare cell growth in cell culture media that is prepared and/or cultured according to three different methods (methods 1, 2 and 3).

In method 1, a cell culture media was used to culture cells in an incubator in which a controlled atmosphere having an O₂ concentration of 21%, a CO₂ concentration of 5% and a temperature of 37° C. was provided. In method 1, the cell culture media was not prepared by exposure to a controlled atmosphere and the O₂ concentration in the cell culture media prior to cell culturing was 10%.

In method 2, a cell culture media was used to culture cells in an incubator in which a reduced oxygen atmosphere having an O₂ concentration of 2%, a CO₂ concentration of 5% and a temperature of 37° C. was provided. In method 2, the cell culture media was not prepared by exposure to a controlled atmosphere. The O₂ concentration in the cell culture media prior to cell culturing was 10%.

In method 3, a cell culture media was used to culture cells in an incubator in which a reduced O₂ atmosphere having an O₂ concentration of 2%, a CO₂ concentration of 5% and a temperature of 37° C. was provided. In method 3, the cell culture was prepared by exposure of the cell culture media to a controlled atmosphere having an O₂ concentration of 2%. The O₂ concentration in the cell culture media prior to cell culturing was 2%.

In methods 1, 2 and 3, the cells cultured were adherent stromal cells recovered from human bone marrow aspirate. The cells were cultured for a period of two weeks for each method. The cell culture used for each method was DMEM.

FIG. 8a shows the number of colony forming units cultured according to methods 1, 2 and 3. FIG. 8b shows the number of cells recovered per 75 cm² from the cell culture media after culturing according to methods 1, 2 and 3. FIG. 8c shows the average number of cells per colony forming unit according to methods 1, 2 and 3.

FIGS. 8a to 8c suggest that the number of cells and colony forming units can be significantly increased by culturing cells in an atmosphere having a reduced O₂ concentration. This result is supported by previous studies which suggest that it is beneficial to culture cells in hypoxic conditions [1, 2, 6, 7, 8, 9].

FIGS. 8a to 8c also suggest that the number of cells and colony forming units can be significantly increased by reducing the O₂ concentration in the cell culture media before cells are cultured in the media. In other words, by using a cell culture media having a reduced O₂ content to culture cells, the number of colony forming units and cells which are cultured can be increased.

One of ordinary skill after reading the foregoing description will be able to affect various changes, alterations, and subtractions of equivalents without departing from the broad concepts disclosed. It is therefore intended that the scope of the patent granted hereon be limited only by the appended claims, as interpreted with reference to the description and drawings, and not by limitation of the embodiments described herein.

The following statements provide general expressions of the disclosure herein.

A. A refrigerated device for exposing containers of liquid media to a defined atmosphere, said device being sealable so as to provide an essentially gas-tight chamber, characterised in that the device is provided with means for supplying one or more gases and means for detecting the concentration of O₂, such that a predetermined concentration of O₂ may be maintained within the chamber.
B. The device according to statement A, wherein the one or more gases supplied are selected from the list consisting of N₂, CO₂, O₂ and an inert gas.
C. The device according to either statement A or B, wherein the O₂ within the chamber may be maintained at less than normal atmospheric concentration.
D. The device according to statement C, wherein the O₂ concentration may be maintained at less that 10%.
E. The device according to statement D, wherein the O₂ concentration may be maintained at less than 5%.
F. The device according to statement E, wherein the O₂ concentration may be maintained at less than 3%.
G. The device of any preceding statement, further comprising a CO₂ detector and means for supplying CO₂, such that a predetermined concentration of CO₂ may be maintained within the chamber.
H. The device of any preceding statement, comprising means by which the pressure of gas within the chamber may be reduced to below atmospheric pressure.
I. The device of any preceding statement comprising a display providing a read-out of the internal concentration of O₂.
J. The device of statement I wherein the display further provides a readout of one or more of: temperature, concentration of CO₂, concentration of N₂, or relative humidity.
K. A method of cell culture comprising the step of pre-equilibrating cooled medium with O₂ at a concentration less than normal atmospheric concentration.
L. A method according to statement K, wherein the medium is cooled to 10° C. or less before or during the pre-equilibration step.
M. A refrigerated device for exposing containers of liquid media to a defined atmosphere, essentially as described herein with reference to FIG. 1.

### REFERENCES

1. Forsyth N R, Musio A, Vezzoni P, Simpson A H, Noble B S, McWhir J. Physiologic oxygen enhances human embryonic stem cell clonal recovery and reduces chromosomal abnormalities. Cloning Stem Cells 2006; 8(1):16-23.
2. Forsyth N R, Evans A P, Shay J W, Wright W E. Developmental differences in the immortalization of lung fibroblasts by telomerase. Aging Cell 2003 October; 2(5):235-43.
3. Ebbesen P, Pettersen E O, Denekamp J, Littbrand B, Keski-Oja J, Schousboe A, Sonnewald U, Amellem O, Zachar V. Hypoxia, normoxia and hyperoxia—terminology for medical in vitro cell biology. Acta Oncol 2000; 39(2):247-8.
4. Ramirez-Bergeron D L, Simon M C. Hypoxia-inducible factor and the development of stem cells of the cardiovascular system. Stem Cells 2001; 19(4):279-86.
5. Semenza G L. Regulation of hypoxia-induced angiogenesis: a chaperone escorts VEGF to the dance. J Clin Invest 2001 July; 108(1):39-40.
6. D'Ippolito G, Howard G A, Roos B A, Schiller P C. Isolation and characterization of marrow-isolated adult multilineage inducible (MIAMI) cells. Exp Hematol 2006 November; 34(11):1608-10.
7. Ezashi T, Das P, Roberts R M. Low O2 tensions and the prevention of differentiation of hES cells. Proc Natl Acad Sci USA 2005 March; 102(13):4783-8.
8. Ludwig T E, Levenstein M E, Jones J M, Berggren W T, Mitchen E R, Frane J L, Crandall L J, Daigh C A, Conard K R, Piekarczyk M S, Lianas R A, Thomson J A. Derivation of human embryonic stem cells in defined conditions. Nat Biotechnol 2006 February; 24(2):185-7.
9. Martin J A, Klingelhutz A J, Moussavi-Harami F, Buckwalter J A. Effects of oxidative damage and telomerase activity on human articular cartilage chondrocyte senescence. J Gerontol A Biol Sci Med Sci 2004 April; 59(4):324-37.
10. Parmar K, Mauch P, Vergilio J A, Sackstein R, Down J D. Distribution of hematopoietic stem cells in the bone marrow according to regional hypoxia. Proc Natl Acad Sci USA 2007 March; 104(13):5431-6.
11. Allen C B, Schneider B K, White C W. Limitations to oxygen diffusion and equilibration in in vitro cell exposure systems in hyperoxia and hypoxia. Am J Physiol Lung Cell Mol Physiol 2001 October; 281(4):L1021-L1027.
12. “Calculating the Oxygen Diffusion Coefficient in Air, Tom Richard, http://www.css.cornell.edu/compost/oxygen/oxygen.diff.air.html
13. “Calculating the Oxygen Diffusion Coefficient in Water, Tom Richard, http://www.css.cornell.edu/compost/oxygen/oxygen.diff.water.html

1. A method of preparing cell culture media for use in culturing cells, the method including:
providing a vessel containing cell culture media; and
exposing the cell culture media to a first controlled atmosphere having a predetermined O₂ concentration that is less than an external atmospheric O₂ concentration, to reduce the O₂ concentration in the cell culture media before culturing cells in the cell culture media.

2. A method according to claim 1 wherein the method includes agitating the cell culture media in the first controlled atmosphere.

3. A method according to claim 2 wherein the cell culture media is agitated by rotation, sonication, vibration and/or stirring of the cell culture media.

4. A method according to claim 2 wherein the step of agitating the cell culture media is performed over a period of at least 1 minute, 1 hour, 3 hours, 6 hours, 12 hours, or 24 hours.

5. A method according to claim 1 wherein the O₂ concentration in the cell culture media before exposing the cell culture media to the first controlled atmosphere is 4% or more, 6% or more, 8% or more, or 10% or more.

6. A method according to claim 1 wherein the O₂ concentration in the cell culture media after exposing the cell culture media is 10% or less, 8% or less, 6% or less, 5% or less, 4% or less, or 2% or less.

7. A method according to claim 1 wherein the first controlled atmosphere has an O₂ concentration of 15% or less, 10% or less, 5% or less, or 2% or less.

8-9. (canceled)

10. A method according to claim 1 wherein the first controlled atmosphere has a predetermined temperature which is less than 37° C. or is 30° C. or less, 20° C. or less, 15° C. or less, 10° C. or less, 5° C. or less, or about 4° C.

11. A method according to claim 1 wherein the first controlled atmosphere has a predetermined pressure which is at least 20 kPa, 40 kPa or 70 kPa below or above an external atmospheric pressure.

12. (canceled)

13. A method according to claim 1 wherein the first controlled atmosphere has a predetermined CO₂ concentration which is more than an external atmospheric CO₂ concentration.

14. A method according to claim 1 wherein the cell culture media is aqueous mammalian cell culture media.

15. (canceled)

16. A method according to claim 1 including sealing the vessel containing the cell culture media after or during exposing the cell culture media to the first controlled atmosphere.

17. (canceled)

18. A method according to claim 1 wherein the method further includes culturing cells in the cell culture media in a second controlled atmosphere after the cell culture media has been exposed to the first controlled atmosphere.

19. A method according to claim 18 wherein the second controlled atmosphere has a predetermined O₂ concentration that is less than an external atmospheric O₂ concentration.

20. A cell culture media preparation apparatus configured to reduce the O₂ concentration in cell culture media before culturing cells in the cell culture media, the apparatus having:
a sealable chamber configured to hold one or more vessels containing cell culture media; and
an atmosphere control unit configured to provide a first controlled atmosphere within the chamber, the first controlled atmosphere having a predetermined O₂ concentration that is less than an external atmospheric O₂ concentration.

21. A cell culture media preparation apparatus according to claim 20 having an agitation unit for agitating cell culture media in the first controlled atmosphere.

22-23. (canceled)

24. A cell culture media preparation apparatus according to claim 20 wherein the atmosphere control unit includes a temperature control unit for controlling the temperature of the first controlled atmosphere and/or a pressure control unit for controlling the pressure of the first controlled atmosphere.

25-27. (canceled)

28. A cell culture media preparation apparatus according to claim 20 wherein the chamber contains a first controlled atmosphere having a predetermined O₂ concentration that is less than an external atmospheric O₂ concentration.

29-31. (canceled)

32. A vessel containing cell culture media having an O₂ concentration which is less than an O₂ concentration in the cell culture media when the cell culture media has been equilibrated with an external atmospheric O₂ concentration.

33-34. (canceled)

35. A method of cell culture, the method including:
(a) providing a vessel containing cell culture media;
exposing the cell culture media to a first controlled atmosphere having a predetermined O₂ concentration that is less than an external atmospheric O₂ concentration, to reduce the O₂ concentration in the cell culture media before culturing cells in the cell culture media; and
agitating the cell culture media;
wherein the first controlled atmosphere has a predetermined temperature that is less than 37° C.; and
(b) culturing cells in the cell culture media in a second controlled atmosphere after the cell culture media has been exposed to the first controlled atmosphere.
